(19) United States
(12) Patent Application Publication
(10) Pub. No.: US 2015/0283353 A1
(43) Pub. Date: Oct. 8, 2015

(54) Restorative Sleep System

(71) Applicant: SUNBEAM PRODUCTS, INC., Boca Raton, FL (US)

(72) Inventors: Gabriel Kohn, Boca Raton, FL (US); Vera Bevini, Somerville, MA (US); Mike Fretwell, Weston, FL (US)

(73) Assignee: Sunbeam Products, Inc., Boca Raton, FL (US)

(21) Appl. No.: 14/441,548

(22) PCT Filed: Oct. 9, 2014
(86) PCT No.: PCT/US2014/032828

§ 371 Date: May 8, 2015

Related U.S. Application Data
(60) Provisional application No. 61/807,838, filed on Apr. 3, 2013.
(60) Provisional application No. 61/874,595, filed on Sep. 6, 2013.

(51) Int. Cl. A61M 21/02; A61F 7/00

(52) U.S. Cl. CPC A61M 21/02; A61F 7/00; A61F 7/0085; A61F 2007/0093; A61M 2021/0066

(57) ABSTRACT

A sleep system including a thermal pad and a thermal control unit in thermal communication with the thermal pad. The thermal controller is configured to automatically adjusts a temperature of the thermal pad during a sleep cycle time according a programmed routine. During the preprogrammed routine, the thermal control unit maintains the temperature in the thermal pad at a first temperature T1. After a first time period S1 from the initiation of the sleep cycle, the thermal control unit adjusts the temperature in the thermal pad a ΔT, for example to a second temperature T2. The second temperature T2 is dependent of the first, preset, temperature T1 and the ΔT. The thermal pad temperature is maintained at the second temperature T2 for a majority of the sleep cycle. At a time period S2 before the end of the sleep cycle, the temperature in the thermal pad 12 adjusted a ΔT to a third temperature T3.

### FIELD OF THE INVENTION

The invention relates generally to heating/cooling pads, and, more particularly, to heating/cooling bedding pads which adjust the temperature during the user's sleep cycle.

### BACKGROUND OF THE INVENTION

Sleeping well is essential to one's physical health and emotional well-being. Even minimal sleep loss can take a toll on your mood, energy, efficiency, and ability to handle stress.

The sleep process is comprised of phases that are governed by the Circadian rhythm. The Circadian rhythm refers to physical events occurring within the body every 24-hour period. The Circadian rhythm is a fundamental property possessed by all organisms and these rhythms are driven by an internal time-keeping system.

One Circadian related physiological phenomenon in humans is the variation in body core temperature during the various sleep phases. In such a process, the body core temperature begins to drop after a person begins to fall asleep. The core temperature remains below normal throughout the night, but begins to increase to normal levels sometime before waking up.

### SUMMARY OF THE INVENTION

The present disclosure is directed to a sleep system including a thermal pad and a thermal control unit in thermal communication with the thermal pad. The thermal controller is configured to automatically adjusts a temperature of the thermal pad during a sleep cycle time according a programmed routine.

During the preprogrammed routine, the thermal control unit maintains the temperature in the thermal pad at a first temperature T1. After a first time period S1 from the initiation of the sleep cycle, the thermal control unit adjusts the temperature in the thermal pad a ΔT, for example to a second temperature T2. The second temperature T2 is dependent of the first, preset, temperature T1 and the ΔT. The second temperature can be less than the first temperature.

The thermal pad temperature is maintained at the second temperature T2 for a majority of the sleep cycle. At a time period S2 before the end of the sleep cycle, the temperature in the thermal pad 12 adjusted a ΔT to a third temperature T3. The third temperature can be greater than the second temperature.

### BRIEF DESCRIPTION OF THE DRAWINGS

A more complete understanding of the present invention, and the attendant advantages and features thereof, will be more readily understood by reference to the following detailed description When considered in conjunction with the accompanying drawings wherein:

FIG. 1 depicts a schematic diagram of the sleep system of the present disclosure;

FIG. 2 depicts an isometric view of the thermal control unit of the sleep system of the present disclosure;

FIG. 3 depicts a graphical representation of a sleep cycle of the present disclosure.

FIG. 4 depicts a flow chart of an exemplary mode of operation of the sleep system of the present disclosure;

FIG. 5 depicts and exemplary control panel of the present disclosure;

FIG. 6 depicts a flow chart of a second exemplary mode of operation of the sleep system of the present disclosure;

FIG. 7 depicts a flow chart of third exemplary mode of operation of the sleep system of the present disclosure; and

FIG. 8 depicts a flow chart of a multi-operational mode of the sleep system of the present disclosure.

### DETAILED DESCRIPTION OF THE INVENTION

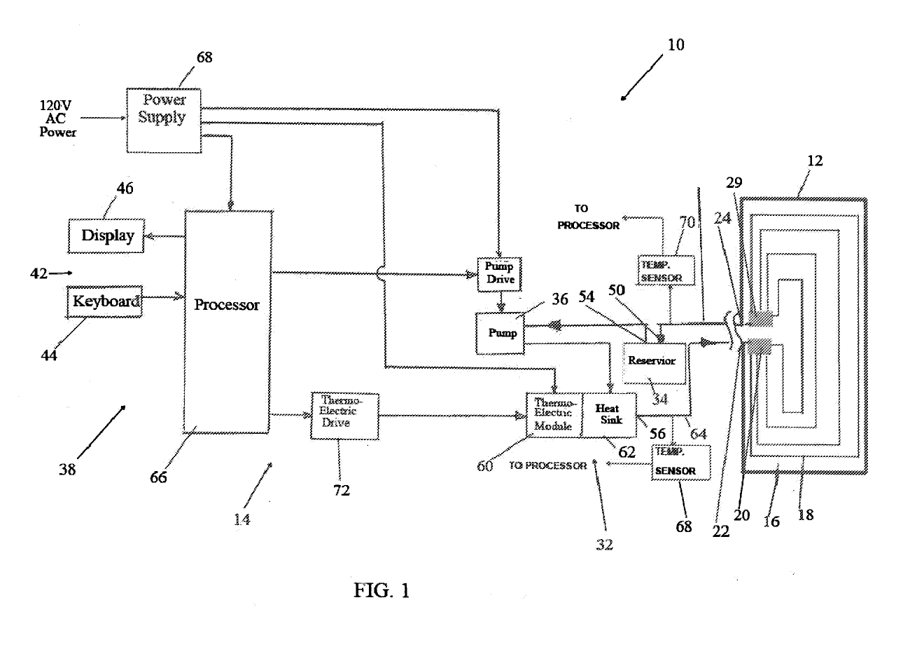

Referring now to the drawing figures in which like reference designators refer to like elements, there is shown in FIG. 1 a sleep system 10 of the present disclosure. The sleep system 10 includes a thermal pad 12 in fluid connection to a thermal control unit 14. The thermal pad 12 is positionable on a bed, such that a user can sleep atop the thermal pad 12. In this position, the thermal pad 12 is capable of selectively cooling or heating the user's body during sleep.

The thermal pad 12 can include a body portion 16 having a serpentine fluid path 18 there though, where the ends 20 of the serpentine fluid path 18 are in fluid communication with a fluid inlet 72 and a fluid outlet 24. In this manner, a fluid can be transported through the serpentine fluid path 18 to heat or cool the thermal pad 12. The fluid can be water, thermal gel, or other thermally conductive fluids. The thermal pad 12 can further include a cover, not shown, positionable over the body portion 16, where the cover is formed of a soft fabric material, and/or can include foam-like-material for comfort.

The serpentine fluid path 18 can he formed by tubing positioned through the body portion 16 in a serpentine pattern. Alternatively, the body portion 16 can be formed from sheets of plastic, which are laminated together to form the serpentine fluid path 18.

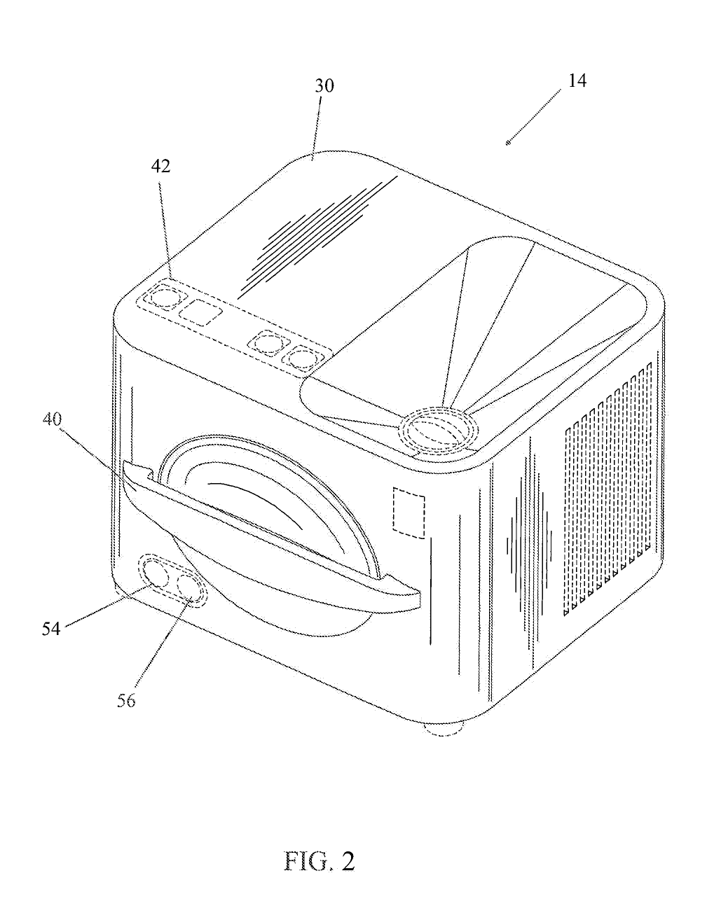

Referring also to FIG. 2, the thermal control unit 14 can take the form of a housing 30 including a heat exchanger 32, fluid reservoir 34, pump 36, and controller 38 positioned therein. The housing 30 can be portable, including a handle 40. A portion of the housing 20 can include a control panel 42 operatively connected to the controller 38, and on which are mounted the operational controls 44 and a display 46. Alternatively, the control panel 42 can be provided as a separate unit, either being connected by wire or wirelessly connected to the controller 38. A wireless controller can take the form of a smart phone, PDA, or other such wireless devices. To enable the thermal control unit 14 to connect to a wirelessly control panel, the controller 38 can further include a wireless transmitter/receiver configured to transmit is various wireless protocol, such as Wi-Fi and BLUETOOTH® protocols.

The reservoir 34 can be configured to how hold an amount of the fluid, and can include a removable fill cap. A fluid inlet portion 50 of the reservoir is connectable to the fluid outlet 24 of the thermal pad 12. The connection of the reservoir 24 to the thermal pad 12 can be performed with a first (return) hose 52, where opposing ends of the first hose 52 can be removeably connected to the reservoir fluid inlet portion 50 and the fluid outlet 24 of the thermal pad 12.

A fluid outlet portion 54 of the reservoir 34 is in fluid communication with the pump 36, where the pump 36 is also connected in fluid communication to the heat exchange 32. The heat exchanger 32 can include a fluid outlet portion 56 connectable to the fluid inlet 22 of the thermal pad 12.

The heat exchanger 32 can take the form of a thermo-electric module 60 connected to a heat sink 62, where the fluid is in thermal communication with the heat sink 62 as it travels through the heater exchanger 32 to heat or cool the fluid. While a thermo-electric heater exchanger is described other heating and cooling elements are also contemplated. The connection of heat exchanger 32 to the thermal pad 12 can be performed with a second (intake) hose 64, where opposing ends of the second hose 64 can be removeably connected to the heat exchanger fluid outlet 56 and the fluid inlet 22 on the thermal pad 12.

In this manner, the pump 36 can transport the fluid from the reservoir 34 through the heat exchanger 32, where the heat exchanger 32 can either heat or cool the fluid. The fluid is pumped from the heat exchanger 32 to and through the serpentine fluid path 18 in the thermal pad 12. The fluid from the thermal pad 12 is then returned to the reservoir 34. This process can act on a continuous base during a sleep cycle, maintaining the desired temperatures in the thermal pad 12.

The controller 38 can include a processor connection 66 to the pump 36 and the heat exchanger 32. The control panel 42 is used to provide inputs to the controller 38, such as the temperature setting, the time setting, and the activation of the unit. A power supply 68 is connected to the micro-processer 66, pump 36, and heat exchanger 32.

Temperature sensors 68, 70 can further be included, being located in the first and second hoses 52, 64. The temperature sensors 68, 70 can provide feedback to the controller 38 regarding the temperature of the fluid entering and exiting the thermal pad 12. Utilizing the temperature sensors 68, 70, the controller 38 can monitor and adjust the temperature of the circulating fluid. The controller 38 can provide a control signal to the heater exchanger 32 based on temperature sensor 68, 70 readings, to heat or cool the circulating fluid as required. To switch from cooling to heating, the output polarity of the power supplied by a thermo-electric drive unit 72 of the heat exchanger 32 is reversed which will cause the thermoelectric modules 60 to begin heating.

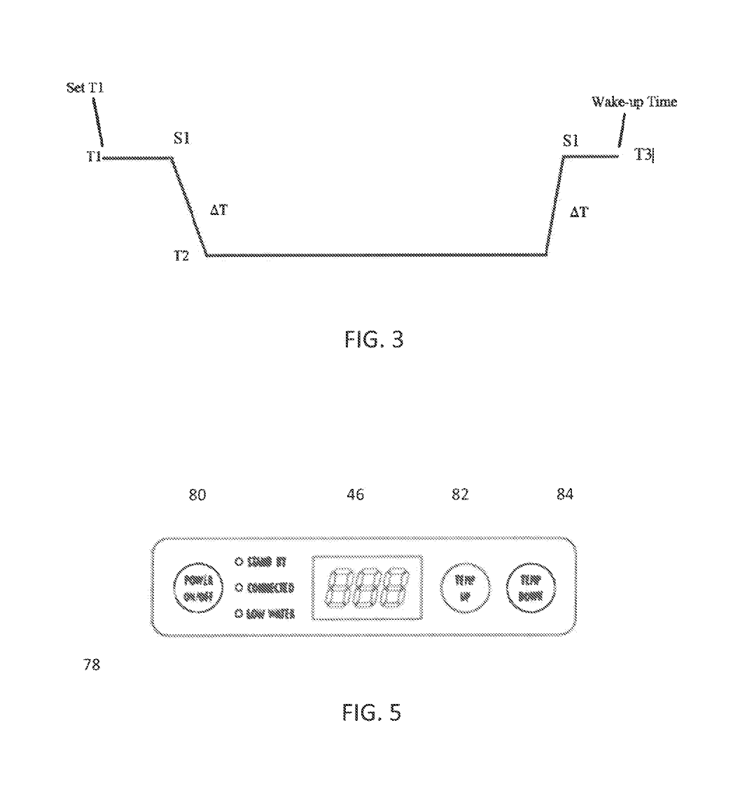

Referring to FIG. 3, during a sleep cycle the control adjusts the temperature of the thermal pad during the sleep cycle. Upon initiation of the sleep cycle, the controller 38 maintains the temperature in the thermal pad 12 at a first temperature T1. The first temperature T1 can be a preset temperature or set by a user. The thermal pad 12 temperature is maintained at this first temperature T1 for a first time period S1. The first time period S1 can be a preset time period or set by a user.

At the end of the first time period S1 the temperature in the thermal pad 12 is decreased ΔT, for example 2-3° F., to a second temperature T2. The decreased ΔT can be a preset temperature or set by a user. The second temperature T2 is dependent of the first, preset, temperature T1 and the ΔT. The temperature change from the first temperature T1 to the second temperature T2 is depicted as a single temperature change ΔT. However, it is contemplated that the temperature change from the first temperature T1 to the second temperature T2 can be achieved using stepped, plurality of discrete, temperature changes ΔT₁, ΔT₂, ΔT₃, . . . , ΔTₙ over a plurality of discrete times S1₁, S1₂, S1₃, . . . , S1ₙ.

The thermal pad 12 temperature is maintained at the second temperature T2 for a majority of the sleep cycle. At a time period S2 before the end of the sleep cycle, the temperature in the thermal pad 12 is increased ΔT to a third temperature T3. The second time period S2 can be a preset time period or set by a user. The second time period S2 can be the same as the first time period S1.

The increased ΔT can be a preset temperature car set by a user. The third temperature T3 is maintained until the sleep cycle ends. It is contemplated that the third temperature T3 is the same as the first temperature T1. It is also contemplated that the third temperature T3 can be different from the first temperature T1. The temperature change from the second temperature T2 to the third temperature T3 is depicted as a single temperature change ΔT. However, it is contemplated that the temperature Change from the second temperature T2 to the third temperature T3 can be achieved using stepped, plurality of discrete, temperature changes ΔT₁, ΔT₂, ΔT₃, . . . , ΔTₙ over a plurality of discrete times S2₁, S2₂, S2₃, . . . , S2ₙ.

In an example of use, the set temperature T1 is 74° F. and the sleep cycle is set at 8 hours. During the sleep cycle, the controller initially maintains the temperature in the thermal pad 12 at the 74° F. set temperature T1. The thermal pad 12 temperature is maintained at this first temperature T1 for a first time period S1, for example 30 minutes. At the end of the first time period S1, the temperature in the thermal pad 12 is decreased a ΔT of 3° F. to a second temperature T2 of 71° F. The thermal pad 12 temperature is maintained at the second temperature T2 for a majority of the sleep cycle. At a time period S2, for example 30 minutes, before the end of the sleep cycle, the temperature in the thermal pad 12 is increased ΔT of 3° F. to a third temperature T3 of 74° F. The third temperature is maintained until the end of the sleep cycle, after which the unit automatically shuts off. Not that the selected temperatures and times are only exemplary in nature, and other temperatures and times are contemplate.

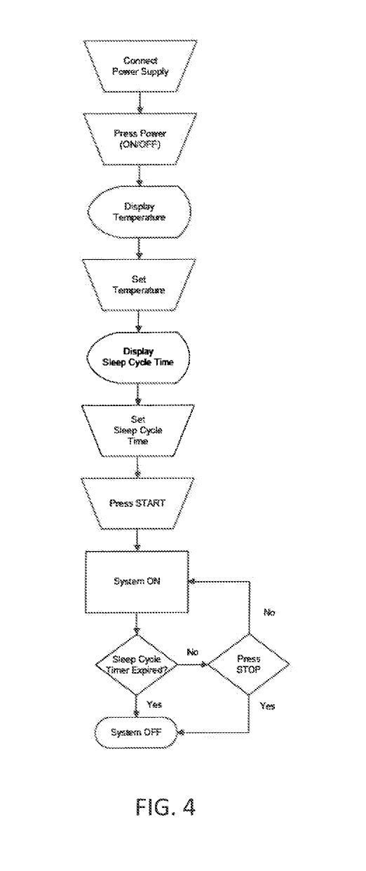

Referring to FIG. 4, a fixed time mode embodiment the sleep system is provided. In the fixed time mode the control panel 42 is utilized to set the first temperature T1 and then a fixed sleep cycle time. Upon connection of the power supply 60 the controller 38 is turned on. The display 46 displays a preset temperature. If desired, the user can change the preset temperature. Upon setting the temperature, a preset sleep cycle time is displayed. If desired, the user can change the preset sleep cycle time. Once initiated, the unit remained operational until the expiration of the sleep cycle time.

Referring to FIG. 5, an exemplary control panel 78 is provided. The control panel 78 can include first 80, second 82, and third buttons 84, and a display 86, where the display 86 can be used to display the set temperature. The first button 82 can be a selector button used to turn the unit on and select a sleep cycle time from a set of sleep cycle times, for example, 4, 5, 7, 8 hours. The continued pressing of the first button 82 can cycle through the set of sleep cycle times, allowing the user to stop on the desired sleep cycle time. The sleep cycle time can initially be preset, for example at 8 hours, and the user has the option to increase or decrease the sleep cycle time.

The second and third buttons 84 and 86 can be used to set the first temperature T1, where the second button 84 can be used to raise the temperature and the third button 86 can be used to lower the temperature. The first temperature T1 can initially be preset, for example at 74° F., and the user has the option to raise or lower this temperature.

In an exemplary method of use of the fixed time mode embodiment, a user utilizes the control panel to set the first temperature T1. For example, the use set the first temperature at 75° F., one degree above a preset first temperature of 74° F. Upon going to bed, the user sets the sleep cycle time. For example, setting the sleep cycle time to 7 hours, one hour less than a preset sleep cycle time of 8 hours. Once the sleep cycle time is set the sleep cycle in initiated.

The controller sets/maintains the thermal pad temperature at the first temperature T1 of 75° F. The temperature is maintained for a first time period S1, for example 20 minutes. At the expiration of the first time period S1, the controller decreases the temperate in the thermal pad by a ΔT, for example 3° F., to a second temperature T2 of 72° F.

At a time period S2, for example 20 minutes, before the end of the set sleep cycle time, the temperature in the thermal pad 12. is increased a ΔT of 3V to a third temperature T3 of 75° F. The third temperature T3 is maintained until the end of the set sleep cycle time, after which the controller automatically turns off power to the pump and heat exchanger. Note that the selected temperatures and times are only exemplary in nature, and other temperatures and times are contemplated.

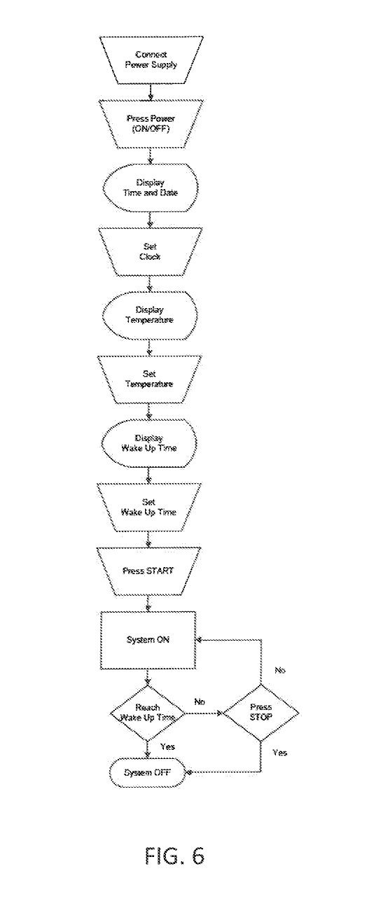

Referring to FIG. 6, a wake-up time mode embodiment of the sleep system is provided, In the wake-up time mode embodiment the control panel 38 is utilized to set the time, a first temperature T1 and a wake-up time. Upon connection of the power supply 68 the controller 38 is turned on. The display 46 displays the time and date. If not set, the user can set the time and date. A preset temperature is then displayed. If desired, the user can change the preset temperature. Upon setting the temperature, the user can then set the wake up time. Once initiated, the unit remained operational until the time reached the wake-up time.

Referring to FIG. 5, an exemplary control panel 78 is provided. The control panel 78 can include first 80, second 82, and third buttons 84, and a display 86, where the display 86 can be used to display the set temperature. The first button 80 can be a selector button used to turn the unit on and used to choose between the time, wake-up-time, and temperature.

In setting the time the up and down buttons 82 and 84 can be used to set the time, including a.m. and p.m. To set the wake-up time, the wake-up-time is selected and the up and down buttons 82 and 84 are used to set the wake-up time, including a.m. and p.m. After the time and the wake-up time are set, the controller automatically determines the sleep cycle time, and sets the time related operating parameters accordingly.

To set the temperature, the user selects the temperature, and utilizes the up and down 82 and 84 buttons set the temperature. Note that the setting of the temperature is optional, as the sleep system 10 can be preprogrammed with a first temperature T1. In addition to being able to select the first temperature, it is also contemplated that the other above noted operating parameter can be adjusted.

In an exemplary method of use, a user utilizes the control panel to set the first temperature T1. For example, the user set the first temperature at 75° F., one degree above a present first temperature of 75° F. Upon going to bed or prior thereto, the user set the wake-up time, 6:30 a.m. To initiate the sleep system the user depresses the start button. Upon initiation, the controller 38 automatically determines the sleep cycle time.

The control system sets/maintains the thermal pad temperature at the first temperature T1 of 75° F. The temperature is maintained for a first time period S1, for example 20 minutes. At the expiration of the first time period S1, the control system decreases the temperate in the thermal pad by a ΔT, for example 3° F., to a second temperature T2 of 72° F.

At a time period S2, for example 20 minutes, before the wake-up-time, the temperature in the thermal pad 12 is increased a ΔT of 3° F. to a third temperature T3 of 75° F., The third temperature T3 is maintained until the wake-up time, after which the unit automatically shuts off. Not that the selected temperature and time are only exemplary in nature, and other temperatures and times are contemplate.

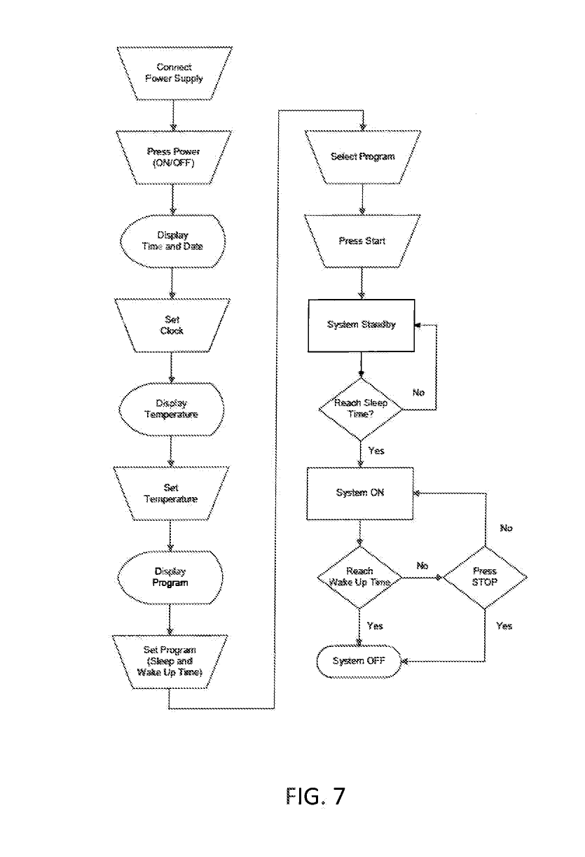

Referring to FIG. 7, a program time mode embodiment of the sleep system is provided. In the program mode the control panel 38 control panel allows a user to set the time, a first temperature T1, start time, and a wake-up time. Upon connection of the power supply 68 the controller 38 is turned on. The display 46 displays the time and date. If not set, the user can set the time and date. A preset temperature is then displayed. If desired, the user can change the preset temperature. Upon setting the temperature, the user can then set the sleep and wake up time. Once initiated, the unit remained operational until the time reached the wake-up time.

Referring to FIG. 5, an exemplary control panel 78 is provided. The control panel 78 can include first 80, second 82, and third buttons 84, and a display 86, where the display 86 can be used to display the set temperature. The first button 80 can be a selector button used to turn the unit on and used to choose between the time, sleep time, wake-up-time, and temperature.

In setting the time the up and down buttons 82 and 84 can be used to set the time, including a.m. and p.m. In setting the time, the up and down buttons 82 and 84 can he used to set the time, including a.m. and p.m. To set the wake-up time, the wake-up-time is selected and the up and down buttons 82 and 84 are used to set the sleep time and wake-up time, including a.m. and p.m. The sleep time can be the time the user plans to go to be, or a set time before and after the user plans to go to bed. After the time, sleep time, and the wake-up times are set, the controller automatically determines the sleep cycle time, and sets the time related operating parameters accordingly.

To set the temperature, the user selects the temperature, and utilizes the up and down 82 and 84 buttons set the temperature. Note that the setting of the temperature is optional, as the sleep system 10 can be preprogrammed with a first temperature T1. In addition to being able to select the first temperature, it is also contemplated that the other above noted operating parameter can be adjusted.

In an exemplary method of use, a user utilizes the control panel to set the first temperature T1. For example, the user set the first temperature at 75° F., one degree above a present first temperature of 75° F. Upon going to bed or prior thereto, the user set the start time and the wake-up time, for example, 9:00 p.m. and 6:30 a.m. The sleep system will automatically start at the set start time.

The control system sets/maintains the thermal pad temperature at the first temperature T1 of 75° F. The temperature is maintained for a first time period S1, for example 20 minutes. At the expiration of the first time period S1, the control system decreases the temperate in the thermal pad by a ΔT, for example 3° F., to a second temperature T2 of 72° F.

At a time period S2, for example 20 minutes, before the wake-up-time, the temperature in the thermal pad 12 is increased a ΔT of 3° F. to a third temperature T3 of 75° F. The third temperature T3 is maintained until the wake-up time, after which the unit automatically shuts off. Not that the selected temperature and time are only exemplary in nature, and other temperatures and times are contemplate.

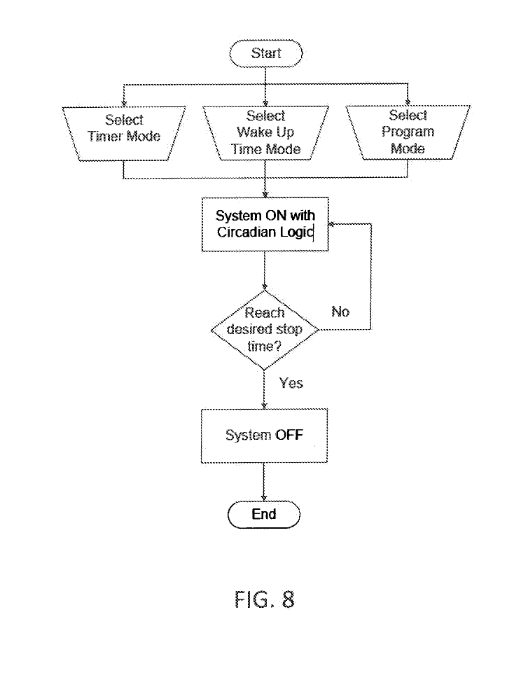

Referring to FIG. 8, a selectable operation mode s provided. In the selectable operation mode, the user can select between the three above described operation mode, namely, the Timer mode, the wake up time mode, and the program time mode.

The above disclosed operational modes are exemplary in nature, and it is contemplated that all of the operational parameters, including the times, sleep cycle times, sleep time, wake-up time, first temperature T1, change in temperature ΔT, third temperature, first time S1 and second time S2 can be adjustable.

All references cited herein are expressly incorporated by reference in their entirety.

All references cited herein are expressly incorporated by reference in their entirety.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A sleep system comprising;
a thermal pad; and
a thermal control unit in thermal communication with the thermal pad, wherein the thermal controller automatically adjusts a temperature of the thermal pad during a sleep cycle time according a programmed routine.

2. The sleep system as set as set forth in claim 1, the thermal control unit comprising a controller including a processor and a control panel, wherein the processor automatically adjusts a temperature of the thermal pad during a sleep cycle time according a programmed routine

3. The sleep system as set forth in claim 2, the thermal control unit further comprising a fluid reservoir and a heat exchanger, wherein an inlet portion of the fluid reservoir is in fluid communication with a outlet portion of the thermal pad, an outlet portion of the fluid reservoir is in fluid communication with an inlet portion of the heat exchange, and an outlet portion of the heat exchanger is in fluid communication with an inlet portion of the thermal pad.

4. The sleep system as set forth in claim 3, wherein the thermal pad comprises a fluid path between the inlet portion of the thermal pad and the outlet portion of the thermal pad.

5. The sleep system as set forth in claim 3, wherein the heater exchanger can increase or decrease the temperature of the thermal pad.

6. The sleep system as set forth in claim 5, wherein the heat exchanger includes a thereto-electric module.

7. The sleep system as set for in claim 1, wherein the sleep cycle time comprises a first time period S1 for which a first temperature T1 is maintaining in the thermal pad, a second time for which a second temperature is maintained in the thermal pad, and third time period for which a third temperature in maintained in the thermal pad.

8. The sleep system as set forth in claim 7, wherein the second temperature is less than the first temperature.

9. The sleep system as set forth in claim 7, wherein the second temperature is greater than the first temperature.

10. The sleep system as set forth is claim 7, wherein the first temperature is equal to the third temperature.

11. The sleep system as set forth in claim 7, wherein the first temperature is different from the third temperature.

12. A method of controlling a sleep system including a thermal pad and a thermal control unit in thermal communication with the thermal pad, comprising the step of
setting a sleep cycle time;
setting a first temperature;
activating the sleep system;
adjusting the first temperature to a second temperature after a first time period;
adjusting the second temperature to a third temperature after a second time period;
turning of the sleep system at the expiration of the sleep cycle time.

13. The method of controlling a sleep system as set forth in claim 12, wherein setting the sleep cycle time comprises;
setting a clock time;
setting a wake-up time; and
calculating the sleep cycle time from the clock time at the time of activation and the wake-up time.

14. The method of controlling a sleep system as set forth in claim 12, wherein setting the sleep cycle time comprises;
setting a clock time;
setting a start time;
setting a wake-up time; and
calculating the sleep cycle time from start time and the wake-up time.

15. The method of controlling a sleep system as set forth in claim 12, wherein the second temperature is less than the first temperature.

16. The method of controlling a sleep system as set forth in claim 12, wherein the second temperature is greater than the first temperature.

17. The method of controlling a sleep system as set forth in claim 12, wherein the first temperature is equal to the third temperature.

18. The method of controlling a sleep system as set forth in claim 12, wherein the first temperature is different from the third temperature.

19. A sleep system comprising;
a thermal pad; and
a thermal control unit in thermal communication with the thermal pad, wherein the thermal controller automatically adjusts a temperature of the thermal pad during a sleep cycle time according a programmed routine,
wherein the sleep cycle time comprises a first time period S1 for which a first temperature T1 is maintaining in the thermal pad, a second time for which a second temperature is maintained in the thermal pad, and third time period for which a third temperature in maintained in the thermal pad, and
wherein the second temperature is less than the first temperature.

20. The sleep system as set forth is claim 19, wherein the third temperature is greater than the second temperature.
